(19) United States
(12) Patent Application Publication
(10) Pub. No.: US 2014/0257007 A1
(43) Pub. Date: Sep. 11, 2014

(54) CHEMICAL REACTION PROCESS AT CONSTANT HYDROGEN HALIDE PARTIAL PRESSURE

(71) Applicant: BASF SE, Ludwigshafen (DE)

(72) Inventors: Stefan Bitterlich, Dirmstein (DE); Steffen Tschirschwitz, Mannheim (DE); Jochen Bürkle, Mannheim (DE); Michael Hübner, Lampertheim (DE)

(73) Assignee: BASF SE, Ludwigshafen (DE)

(21) Appl. No.: 14/199,117

(22) Filed: Mar. 6, 2014

Related U.S. Application Data
(60) Provisional application No. 61/773,842, filed on Mar. 7, 2013.

(51) Int. Cl. C07C 5/27

(52) U.S. Cl. CPC C07C 5/27
USPC 585/734

(57) ABSTRACT

The present invention relates to a chemical reaction process, preferably an isomerization process, of at least one hydrocarbon in the presence of an ionic liquid and a hydrogen halide (HX). The chemical reaction is carried out in an apparatus (V1) in which a gas phase is in direct contact with a liquid reaction mixture. The gas phase and the liquid reaction mixture each comprise the hydrogen halide and the liquid reaction mixture additionally comprises at least one hydrocarbon and the ionic liquid. Gaseous HX is introduced into the apparatus (V1) in such a way that the hydrogen halide partial pressure is kept constant in the gas phase. The ionic liquid used in the respective chemical reaction, in particular in an isomerization, can (inter alia) be regenerated by the process of the invention.

This patent application claims the benefit of pending provisional patent application Ser. No. 61/773,842 filed on Mar. 7, 2013, incorporated in its entirety herein by reference.

The present invention relates to a chemical reaction process, preferably an isomerization process, of at least one hydrocarbon in the presence of an ionic liquid and a hydrogen halide (HX). The chemical reaction is carried out in an apparatus (V1) in which a gas phase is in direct contact with a liquid reaction mixture. The gas phase and the liquid reaction mixture each comprise the hydrogen halide and the liquid reaction mixture additionally comprises at least one hydrocarbon and the ionic liquid. Gaseous HX is introduced into the apparatus (V1) in such a way that the hydrogen halide partial pressure is kept constant in the gas phase. The ionic liquid used in the respective chemical reaction, in particular in an isomerization, can (inter alia) be regenerated by the process of the invention.

Ionic liquids, in particular acidic ionic liquids, are suitable, inter alia, as catalysts for the isomerization of hydrocarbons. Such a use of an ionic liquid is, for example, disclosed in WO 2011/069929 where a specific selection of ionic liquids is used in the presence of an olefin to isomerize saturated hydrocarbons, in particular for isomerizing methylcyclopentane (MCP) to cyclohexane. An analogous process is described in WO 2011/069957 but there the isomerization is not carried out in the presence of an olefin but instead using a copper(II) compound.

In general, ionic liquids and hydrocarbons (organic phases) are immiscible or only very sparingly miscible and form two separate phases. To be able to utilize the above-mentioned catalytic effect, intensive contact has to be established between organic phase and the ionic liquid. For this purpose, the two phases are frequently mixed in stirred vessels with intensive stirring to give dispersions. Depending on parameters such as type of ionic liquid or of ionic phase or the phase ratio, the dispersion can either be present as a dispersion of an ionic liquid in the organic phase or can be a dispersion of the organic phase in the ionic liquid.

Especially in a continuous mode of operation, a partial amount and/or constituents of the ionic liquid used, in particular the anion part, is continually discharged in the form of metal halides such as aluminum chloride and/or hydrogen halide such as HCl via the organic phase in a chemical reaction process, in particular in an isomerization, as a result of which a reduction in the activity of the ionic liquid used, preferably as catalyst, in the chemical reaction process is found.

EP-A 2 455 358 relates to processes for regenerating and maintaining the activity of an ionic liquid used as catalyst, in particular in connection with the preparation of alkylates by means of alkylation reactions. Here, hydrogen halide or halogenated hydrocarbons are added to the catalyst (acidic ionic liquid) in the feed stream during the alkylation reaction. The addition of the hydrogen halide or the halogenated hydrocarbon can also be carried out continuously. Furthermore, EP-A 2 455 358 discloses an analogous process for preparing alkylates by means of an alkylation reaction usnig isobutene and C4-alkenes as feed stream and acidic ionic liquids as catalyst. However, in none of the two processes disclosed in EP-A 2 455 358 is the addition of a hydrogen halide carried out in such a way that the hydrogen halide partial pressure is kept constant in a gas phase over the corresponding reaction mixture.

A. Berenblyum (Applied Catalysis. A: General 315 (2006) 128-134) discloses studies on the catalytic activity of chloroaluminate-comprising ionic liquids in connection with the isomerization of heptane. Here, studies are carried out on the solubility of HCl in the chloroaluminate-comprising ionic liquid and on the distribution of aluminum chloride between chloroaluminate-comprising ionic liquid and heptane. In the system examined, HCl is identified as catalytically active component and aluminum chloride is identified as cocatalyst. The decrease in activity of the chloroaluminate-comprising ionic liquid is attributed to the loss of HCl and the formation of an acid-soluble oil which poisons the catalyst. The experiments are (at least in part) carried out with continuous addition of HCl, but there, too, there is no indication that the hydrogen halide partial pressure is kept constant in a gas phase over the corresponding reaction mixture.

US-A 2010/0065476 discloses a method of measuring and adapting the flow of a halogen-comprising additive in a continuous reactor process, for example in alkylations of olefins or aromatics or in dehydrogenation processes. The halogen-comprising additives can be Brönsted acids such as hydrogen chloride, hydrogen bromide or fluorinated alkanesulfonic acids and also metal halides such as sodium chloride or copper chloride. Furthermore, this document discloses apparatuses for carrying out the corresponding method, which comprise a reactor comprising an ionic liquid, measurement devices for determining the halogen concentration in the reactor outlet and a control system fro controlling the halogen concentration.

In the method according to US-A 2010/0065476, the addition of the halogen-comprising additive is not necessarily restricted to one form, since it is possible to use, for example, hydrogen chloride which is gaseous at room temperature or a solid such as sodium chloride, which can also, for example, be added in dissolved form in the continuous reactor process, as halogen-comprising additive. However, if a gaseous halogen-comprising additive such as hydrogen chloride is used, US-A 2010/0065476 does not give any indication that the partial pressure of, for example, hydrogen chloride has to be kept constant in a gas phase over the respective reaction mixture. Furthermore, the method described in US-A 2010/0065476 comprises continual sampling and halide analysis of the feed stream to the reaction as necessary constituent. This complicated procedure can in principle be dispensed with in the present invention.

US-A 2007/0249485 discloses a process for regenerating acidic ionic liquids which have been used as catalyst, where the appropriate ionic liquid is brought into contact with at least one metal in a regeneration zone in the absence of hydrogen. As metal, it is possible to use, for example, aluminum, gallium or zinc, and the ionic liquid is preferably used for catalyzing Friedel-Crafts reactions. An analogous process is disclosed in US-A 2007/0142217, where the regeneration is additionally carried out in the presence of a Brönsted acid such as hydrogen chloride.

WO 2011/006848 discloses a process for modifying an alkylation unit for HF or sulfonic acid and an alkylation unit for ionic liquids. In this process, the ionic liquid used as catalyst is, inter alia, regenerated by adding hydrogen halide or a haloalkane. However, WO 2011/006848 gives no indication that when using a hydrogen halide, the hydrogen halide partial pressure is kept constant in a gas phase over the respective reaction mixture.

It is an object of the present invention to provide a novel process for the chemical reaction of at least one hydrocarbon in the presence of an ionic liquid, in particular for isomerization of at least one hydrocarbon in the presence of an ionic liquid.

The object is achieved by a chemical reaction process of at least one hydrocarbon in an apparatus (V1) in the presence of an ionic liquid and a hydrogen halide (HX), wherein a liquid reaction mixture comprising at least one hydrocarbon, the hydrogen halide and the ionic liquid and a gas phase comprising the hydrogen halide are present in the apparatus (V1), with the liquid reaction mixture and the gas phase being in direct contact with one another and gaseous hydrogen halide being introduced into the apparatus (V1) in such a way that the hydrogen halide partial pressure in the gas phase is kept constant during the chemical reaction.

A chemical reaction, in particular an isomerization, of hydrocarbons can be carried out in an advantageous way by means of the process of the invention. Owing to the gaseous addition of a hydrogen halide (HX) at a constant hydrogen halide partial pressure in the gas phase, the catalytic activity of the respective ionic liquid is kept largely constant. The effect can be reinforced further when, in addition to the hydrogen halide, preferably hydrogen chloride, a metal halide, in particular aluminum chloride, is added to the ionic liquid present in the apparatus (V1) or this ionic liquid is continually in contact with the metal halide.

The process of the invention can be carried out in a particularly simple and thus advantageous way when the hydrogen halide partial pressure in the apparatus (V1) is kept constant by the pressure in the apparatus (V1) being regulated by gaseous hydrogen halide being introduced repeatedly or continuously into the apparatus (V1). In this embodiment, the gaseous hydrogen halide is preferably introduced from a reservoir into the apparatus (V1), with a shut-off device, preferably a valve or a tap, being present between the apparatus (V1) and the reservoir. The pressure in the gas phase (over the reaction mixture) in the apparatus (V1) can thus be measured continuously using relatively simple apparatus, with the shut-off device being opened when the pressure goes below a (prescribed) threshold value and the shut-off device being in turn closed when the pressure exceeds the threshold value.

Furthermore, it is advantageous for (in addition to the introduction of hydrogen halide into the apparatus (V1)) the metal halide not to be added directly to the ionic liquid in the apparatus (V1) but for the metal halide instead to firstly be premixed with a main component present in the apparatus (V1) outside the apparatus (V1) in an apparatus or device (V2). This can, as one alternative, be the ionic liquid itself which originates from the reaction outlet from the apparatus (V1) and is separated off from the reaction outlet in a phase separation unit, preferably a phase separator, and is recirculated to the apparatus (V1).

However, it is particularly advantageous to add the metal halide to the feed stream comprising the hydrocarbons which are to be subjected to a chemical reaction, in particular an isomerization, in the apparatus (V1). In this variant, the apparatus required for addition of the metal halide is simpler because the corresponding apparatus (V2), detached from its specific function, does not have to be made of corrosion-stable material, which is generally necessary in the case of addition to the recirculated ionic liquid or introduction directly into the apparatus (V1) since many ionic liquids are highly corrosive. Furthermore, in the case of addition of the metal halide to the hydrocarbon-comprising stream it is also not necessary for the corresponding apparatus to be designed for high reaction pressures.

The inventive chemical reaction process of at least one hydrocarbon in the presence of an ionic liquid at a constant hydrogen halide partial pressure in the gas phase in the apparatus (V1) is defined in more detail below.

For the purposes of the present invention, a “chemical reaction process” or “chemical reaction” is in principle any chemical reaction known to those skilled in the art in which at least one hydrocarbon is chemically reacted, modified or changed with regard to its composition or structure in another way.

The chemical reaction process is preferably selected from among an alkylation, a polymerization, a dimerization, an oligomerization, an acylation, a metathesis, a polymerization or copolymerization, an isomerization, a carbonylation and combinations thereof. Alkylations, isomerizations, polymerizations, etc., are known to those skilled in the art. For the purposes of the present invention, the chemical reaction process is particularly preferably an isomerization.

For the purposes of the present invention, the chemical reaction, preferably the isomerization, is carried out in an apparatus (V1) known to those skilled in the art. Suitable apparatuses (V1) are, for example, reactors, other reaction apparatuses, stirred vessels or a cascade of stirred vessels. The apparatus (V1) is preferably a reactor or a cascade of stirred vessels.

In principle, any hydrocarbons can be comprised in the apparatus (V1) in the process of the invention. A person skilled in the art will know on the basis of general technical knowledge which hydrocarbons and which compositions are best suited to which specific chemical reaction process. Compounds which themselves are not hydrocarbons can optionally also be comprised (in the form of mixtures). In the following text, the composition of the hydrocarbons comprised in the apparatus (V1) will be illustrated by the isomerization which is preferred as chemical reaction for the purposes of the present invention.

In the chemical reaction in the apparatus (V1), in particular in the isomerization, methylcyclopentane (MCP) or a mixture of methylcyclopentane (MCP) with at least one further hydrocarbon selected from among cyclohexane, n-hexane, isohexanes, n-heptane, isoheptanes, methylcyclohexane and dimethylcyclopentanes is preferably used as hydrocarbon. The corresponding hydrocarbons are thus fed into the apparatus (V1).

A mixture of methylcyclopentane (MCP) with at least one further hydrocarbon selected from among cyclohexane, n-hexane, isohexanes, n-heptane, isoheptanes, methylcyclohexane and dimethylcyclopentanes is more preferably used in the chemical reaction, in particular in the isomerization, with the concentration ratio of MCP/cyclohexane preferably being at least 0.2.

According to the present invention, particular preference is given to isomerizing methylcyclopentane (MCP) to cyclohexane.

Cyclohexane or a mixture of cyclohexane with at least one further hydrocarbon selected from among methylcyclopentane (MCP), n-hexane, isohexane, n-heptane, isoheptane, methylcyclohexane and dimethylcyclopentane is preferably obtained as hydrocarbon after the chemical reaction, in particular after the isomerization, in the process of the invention.

A mixture of cyclohexane, MCP and at least one further hydrocarbon is particularly preferably obtained after the chemical reaction, in particular after the isomerization. The further hydrocarbon is preferably selected from among n-hexane, isohexane, n-heptane, isoheptane, methylcyclohexane and dimethylcyclopentane. Furthermore, in the process of the invention, preference is given to a smaller proportion of MCP and open-chain linear hydrocarbons being present after the isomerization in the mixture obtained, which is preferably comprised in the phase (B) described below, compared to the corresponding composition of the hydrocarbons or the phase (B) before the isomerization.

For the purposes of the present invention, all ionic liquids known to those skilled in the art are in principle suitable as ionic liquids. An overview of suitable ionic liquids may, for the case of isomerization, be found in, for example, WO 2011/069929. For the purposes of the present invention, preference is given to an acidic ionic liquid.

For the purposes of the present invention, preference is given to using (acidic) ionic liquids in which the anion comprises at least one metal component and at least one halogen component.

For the purposes of the present invention, the ionic liquid is preferably used as catalyst in a chemical reaction, preferably in an alkylation or isomerization, in particular in an isomerization. In addition, it can have a solvent capability for another catalyst used in the respective reaction.

In the (preferably acidic) ionic liquid in the process of the invention, the metal component in the anion of the ionic liquid is preferably selected from among Al, B, Ga, In, Fe, Zn and Ti and/or the halogen component is selected from among F, Cl, Br and I, in particular from among Cl and Br. The (preferably acidic) ionic liquid more preferably has a haloaluminate ion having the composition AlₙX₍₃ₙ₊₁₎ where 1<n<2.5 and X=halogen, preferably X═F, Cl, Br or I, in particular X═Cl, as anion.

All cations known to those skilled in the art are in principle suitable as cations. Examples are an unsubstituted or at least partially alkylated ammonium ion or a heterocyclic (monovalent) cation optionally having alkyl side chains, in particular a pyridinium ion, an imidazolium ion, a pyridazinium ion, a pyrazolium ion, an imidazolinium ion, a thiazolium ion, a triazolium ion, a pyrrolidinium ion, an imidazolidinium ion or a phosphonium ion. The at least partially alkylated ammonium ion preferably comprises one, two or three alkyl radicals (each) having from 1 to 10 carbon atoms. If two or three alkyl substituents are present on the respective ammonium ions, the chain length in each case can be selected independently; preference is given to all alkyl substituents having the same chain length. Particular preference is given to trialkylated ammonium ions having a chain length of from 1 to 3 carbon atoms. The heterocyclic cation is preferably an imidazolium ion or a pyridinium ion.

The ionic liquid preferably has an ammonium ion, more preferably trialkylammonium, as cation and/or a chloroaluminate ion of the composition AlₓCl₃ₓ₊₁ where 1<x<2.5 as anion.

The ionic liquid, in particular the acidic ionic liquid, particularly preferably comprises an at least partially alkylated ammonium ion as cation and a chloroaluminate ion having the composition AlₙCl₍₃ₙ₊₁₎ where 1<n<2.5 as anion. Examples of such particularly preferred ionic liquids are tetramethylammonium chloroaluminate and triethylammonium chloroaluminate.

As hydrogen halide (HX), it is in principle possible to use all conceivable hydrogen halides, for example hydrogen fluoride (HF), hydrogen chloride (HCl), hydrogen bromide (HBr) or hydrogen iodide (HI). The hydrogen halides can optionally also be used as a mixture, but, for the purposes of the present invention, preference is given to using only one hydrogen halide.

Preference is given to using the hydrogen halide (HX) whose halogen component (X) is (at least partly) the same as the halogen component in the anion of the above-described (acidic) ionic liquid. The hydrogen halide (HX) is preferably hydrogen chloride (HCl) or hydrogen bromide (HBr). The hydrogen halide (HX) is particularly preferably hydrogen chloride (HCl). Furthermore, the hydrogen halide (HX) is preferably dry; in particular, the hydrogen halide is dry hydrogen chloride.

A liquid reaction mixture comprising (as components) at least one hydrocarbon, the hydrogen halide (HX) and the ionic liquid is present in the apparatus (V1). The individual components of the liquid reaction mixture correspond to the above definitions, and 2 or more hydrogen halides and/or ionic liquids can optionally also be comprised therein. In other words, the liquid reaction mixture is made up of the components which (actively) participate in the above-described chemical reaction, preferably in the isomerization.

In the apparatus (V1) and in particular in the liquid reaction mixture, the ionic liquid is used as catalyst and the hydrogen halide is used as cocatalyst in a chemical reaction, preferably in an alkylation or isomerization, in particular in an isomerization.

Furthermore, a gas phase comprising the hydrogen halide (HX) is present in the apparatus (V1). The hydrogen halide in the gas phase and the hydrogen halide in the liquid reaction mixture are the same as regards the chemical definition.

The liquid reaction mixture and the gas phase are in direct contact with one another in the apparatus (V1). Here, the liquid reaction mixture generally forms one, two or even more separate phases, i.e. phases which are different from the gas phase. The liquid reaction mixture and the gas phase can, for example, be present as physically separate phases, i.e., for example, the liquid reaction mixture consisting of one or two separate phases is present in the lower part of the apparatus (V1) while the gas phase is in turn present in the upper part of the respective apparatus (V1). At the interface between liquid reaction mixture and the gas phase, there is thus direct contact between the two “main phases” under consideration (i.e. liquid reaction mixture and gas phase). It is also possible for the liquid reaction mixture and the gas phase to be mixed, for example by means of intensive stirring, but with the separation into reaction mixture and gas phase being maintained.

For the purposes of the present invention, at least one hydrogen halide (HX), preferably hydrogen chloride (HCl), is introduced in gaseous form into the apparatus (V1). Owing to the gaseous introduction of the hydrogen halide, the above-described gas phase is formed in the apparatus (V1). The gaseous introduction of the hydrogen halide is carried out in such a way that the hydrogen halide partial pressure in the gas phase is kept constant during the chemical reaction, in particular during the isomerization.

For the purposes of the present invention, the term “constant hydrogen halide partial pressure” (in the gas phase) has the following meaning. The hydrogen halide partial pressure (in the gas phase) is constant when, during the course of the operating time of the apparatus (V1), it deviates by not more than 20%, preferably not more than 10%, more preferably not more than 5%, in particular not more than 1%, from the average value determined over the operating time of the apparatus (V1). Here, the operating time is the normal, correct operation of the apparatus (V1) without taking into account start-up or running-down procedures in the apparatus (V1) and also operational malfunctions.

For the purposes of the present invention, the hydrogen halide partial pressure (p_{HX}) is defined as follows:

p_{HX}=x_{HX}pₜₒₜₐₗ (equation 1)

where
x_{HX}=mole fraction of the hydrogen halide in the gas phase and
pₜₒₜₐₗ=total pressure of the gas phase over the reaction mixture.

In the case of no or only negligibly small amounts of substances apart from hydrogen halide and hydrocarbon being present in the gas phase over the reaction mixture, the hydrogen halide partial pressure p_{HX} is calculated as follows:

p_{HX}=pₜₒₜₐₗ−p_{HC} (equation 2)

where
pₜₒₜₐₗ=total pressure of the gas phase over the reaction mixture,
p_{HC}=vapor pressure of the hydrocarbons of the reaction mixture at the reaction temperature

The hydrogen halide partial pressure p_{HX} can preferably be determined by taking a sample consisting of a defined amount from the gas phase in the apparatus (V1) and determining the HX mole fraction by a method known to those skilled in the art (e.g. passing the gas into a defined NaOH solution and subsequent back-titration) and multiplying the mole fraction determined in this way by the total pressure of the gas phase in (V1) according to equation 1.

However, the hydrogen halide partial pressure p_{HX} can optionally also be estimated according to equation 2 if p_{HC} is known. P_{HC} and also pₜₒₜₐₗ can be determined by methods known to those skilled in the art, in particular measurement of the pressure pₜₒₜₐₗ by means of a conventional pressure measurement device and determination of p_{HC} by means of a temperature-vapor pressure correlation (vapor pressure curve) which is generally known for a given hydrocarbon or a hydrocarbon mixture or can be determined by measurement methods known to those skilled in the art.

The hydrogen halide partial pressure in the gas phase can in principle take on any values in the process of the invention. The hydrogen halide partial pressure in the gas phase is preferably in the range from 1.1 to 5 bara, more preferably from 2 to 4 bara.

In a preferred embodiment of the present invention, the hydrogen halide partial pressure in the gas phase is kept constant by regulating the pressure in the apparatus (V1) by repeated or continuous introduction of gaseous hydrogen halide into the apparatus (V1).

For the purposes of the present invention, “continuous introduction of gaseous hydrogen halide” means that the corresponding addition is effected over a relatively long period of time, preferably over at least 50%, more preferably over at least 70%, even more preferably over at least 90%, of the reaction time, in particular over the entire reaction time. The continuous introduction is preferably carried out so that the corresponding apparatus for the gaseous introduction (addition) of the hydrogen halide is in operation over the abovementioned periods of time.

For the purposes of the present invention, “repeated introduction of gaseous hydrogen halide” means that the corresponding gaseous introduction (addition) is effected at regular or irregular time intervals. The periods of time between the individual additions are at least one hour, preferably at least one day. For the purposes of the present invention, the expression “repeated” also means at least two, for example 3, 4, 5, 10 or even 100, individual additions. The actual number of individual additions depends on the operating time. This ideally tends toward infinity.

In other words, a repeated introduction of gaseous hydrogen halide is, for the purposes of the present invention, the addition separated over time of a plurality of partial amounts of metal halide. The addition of an individual partial amount can take from a number of seconds to a number of minutes; somewhat longer periods of time are optionally also conceivable. According to the invention, the time interval between the respective addition of an individual partial amount is at least ten times as great as the duration of the addition of the corresponding partial amount. For the purposes of the present invention, the embodiment of a “repeated addition” can optionally also be combined with the embodiment of a “continuous addition”.

Furthermore, preference is given to the gas phase in the apparatus (V1) being connected via a shut-off device to a reservoir, where the contents of the reservoir comprise at least 90 mol %, particularly preferably more than 98 mol %, of the hydrogen halide and the reservoir has a pressure which is greater than the hydrogen halide partial pressure of the gas phase in the apparatus (V1).

In this embodiment, the gaseous hydrogen halide is preferably introduced from a reservoir into the apparatus (V1), with a shut-off device, preferably a valve or a tap, being located between the apparatus (V1) and the reservoir. The pressure in the gas phase (over the reaction mixture) in the apparatus (V1) can thus be measured with a relatively simple outlay in terms of apparatus either repeatedly or preferably continuously, with the shut-off device being opened when the pressure goes below a (prescribed) threshold value, while the shut-off device is in turn closed when the pressure exceeds the threshold value.

Furthermore, in an embodiment of the present invention, the pressure in the apparatus (V1) is preferably kept constant by using a two-point regulating system which acts on a shut-off device to a hydrogen halide reservoir.

In a preferred embodiment of the present invention, the apparatus (V1) comprises, in addition to the gaseous phase, two further phases (A and B) which together form the liquid reaction mixture. Further phases can optionally also be comprised in the liquid reaction mixture. Here, phase (A) comprises at least one ionic liquid as per the above description, with the proportion of ionic liquid in the phase (A) being greater than 50% by weight. The phase (A) is preferably a phase which comprises ionic liquids and is immiscible or only sparingly miscible with hydrocarbons and/or comprises not more than 10% by weight of hydrocarbons. In general, the hydrogen halide (HX) is comprised both in the phase (A) and in the phase (B).

For example, mixtures of two or more ionic liquids can be comprised in the phase (A); the phase (A) preferably comprises one ionic liquid. Apart from the ionic liquid, further components which are miscible with the ionic liquid can also be comprised in the phase (A). These can be hydrocarbons from the phase (B) described below which generally have limited solubility in ionic liquids. Furthermore, phase (A) can also comprise cocatalysts which are used in isomerization reactions using ionic liquids. A preferred example of such cocatalysts is the abovementioned hydrogen halides, in particular hydrogen chloride. In addition, constituents or decomposition products of ionic liquids, which can be formed, for example, during the isomerization process, can be comprised in phase (A). The proportion of ionic liquid in phase (A) is preferably greater than 80% by weight.

Phase (B) comprises, for the purposes of the present invention, at least one hydrocarbon, with the content of hydrocarbon in the phase (B) being greater than 50% by weight. Phase (B) is preferably a hydrocarbon-comprising phase which is immiscible or only sparingly miscible with ionic liquids and/or comprises not more than 1% by weight of ionic liquids (based on the total weight of the phase).

The actual composition of the phase (B) depends on the chemical reaction process selected. The phase (B) experiences a change in its composition during the course of a chemical reaction process. The particular hydrocarbons which can be comprised in the phase (B) both before and after the chemical reaction, in particular the isomerization, have been described above.

Furthermore, preference is given to the ionic liquid in the apparatus (V1) being comprised to an extent of greater than 50% by weight in a phase (A) which has a higher viscosity than a phase (B) in which at least one hydrocarbon is comprised to an extent of greater than 50% by weight and the phases (A) and (B) being in direct contact with one another, for example by forming a heterogeneous mixture with one another.

In an embodiment of the present invention, the chemical reaction, in particular the isomerization, occurs in a dispersion (D1) in which the phase (B) is dispersed in the phase (A). The dispersion direction (i.e. the information as to which phase is present in disperse form in the other phase) can be determined by examining a sample, optionally after addition of a dye which selectively colors one phase, in transmitted light under an optical microscope. Here, the phases (A) and (B) have the above definitions.

The dispersion (D1) can be produced by methods known to those skilled in the art; for example, such a dispersion can be produced by intensive mixing of the phases by stirring. In a further embodiment of the present invention, the volume ratio of phase (A) to phase (B) in the dispersion (D1) is in the range from 2.5:1 to 4:1 [vol/vol], preferably in the range from 2.5:1 to 3:1 [vol/vol].

Furthermore, in a preferred embodiment of the process of the invention, at least one metal halide is added to the apparatus (V1) during the chemical reaction, preferably during the isomerization. The addition of the metal halide is thus carried out in addition to the introduction (addition) of the gaseous hydrogen halide (HX). The addition of the metal halide to the apparatus (V1) can be carried out repeatedly or continuously.

The anion of the ionic liquid and the metal halide preferably have the same halogen component and metal component. In principle, all metal halides which are known to those skilled in the art and satisfy this criterion are suitable. The metal halide is preferably selected from among AIX₃, BX₃, GaX₃, InX₃, FeX₃, ZnX₂ and TiX₄ where X=halogen, preferably X=Cl or Br, even more preferably X=Cl. In particular, the metal halide is AlCl₃.

Furthermore, preference is given to the halogen components of ionic liquid, the hydrogen halide (HX) and the metal halide being the same.

If, for example, the ionic liquid used in the apparatus (V1) comprises Al₂Cl₇⁻ as anion, AlCl₃ can correspondingly be used as metal halide. In the case of mixed-component anions such as Al₂BrCl₆⁻, it is possible to use, for example, a corresponding mixture of AlCl₃ and AlBr₃. This also applies analogously in respect of the choice of the appropriate metal component of the metal halide used when the metal component of the anion of the respective ionic liquid comprises two or more components such as Al or Cu.

The addition of at least one metal halide to the apparatus (V1) can be carried out repeatedly or continuously. Here, the metal halide an be added in liquid or solid form. It has also been found that the metal halide does not have to be introduced directly into the apparatus (V1) but the metal halide can instead firstly be added to one or more of the components participating in the chemical reaction process in another apparatus, for example in a contact apparatus (V2). From this other apparatus, the metal halide is conveyed together with the component(s) mentioned into the apparatus (V1) (indirect addition of the metal halide to (V1)). The transfer or conveying of the metal halide together with the component(s) mentioned from the other apparatus into the apparatus (V1) is effected by the methods known to those skilled in the art, for example using pumps.

The two embodiments defined in more detail in the following text in combination with the FIGS. 1 and 2 are preferred for the addition of the metal halide. Both embodiments are an indirect addition in which the metal halide is firstly introduced into the system via the contact apparatus (V2) from where it goes into the apparatus (V1).

For the purposes of the present invention, “continuous addition” of the metal halide means that the corresponding addition occurs over a relatively long period of time, preferably over at least 50%, more preferably over at least 70%, even more preferably over at least 90%, of the reaction time, in particular over the entire reaction time. The continuous addition is preferably carried out with the corresponding apparatus for introduction (addition) of the metal halide (e.g. a star feeder) being in operation over the abovementioned periods of time.

For the purposes of the present invention, “repeated addition” of the metal halide means that the corresponding addition is carried out at regular or irregular time intervals. The corresponding addition is preferably triggered by the occurrence of an addition condition described below, in particular in connection with the saturation concentration in the phase (B). The time intervals between the individual additions are at least one hour, preferably at least one day. For the purposes of the present invention, the term “repeated” again means at least two, for example 3, 4, 5, 10 or even 100, individual additions. The actual number of the individual additions depends on the operating time. This ideally tends toward infinity.

In other words, repeated addition of the metal halide means, for the purposes of the present invention, the addition at separate times of a number of batches of metal halide. The addition of an individual batch can take from a number of seconds to a number of minutes, and somewhat longer periods are optionally also considerable. According to the invention, the time interval between the respective addition of an individual batch is at least ten times as great as the duration of the addition of an individual batch. For the purposes of the present invention, the embodiment of “repeated addition” can optionally be combined with the embodiment of “continuous addition”.

For the purposes of the present invention, the addition of the metal halide is particularly preferably carried out in such a way that a concentration of ≧70%, preferably ≧90%, of the saturation concentration of the metal halide is established in the apparatus (V1). It is also possible for supersaturation of metal halide to occur in the apparatus (V1). If this is the case, an (additional) solid phase of metal halide is formed in the apparatus (V1). A concentration of ≧70% by weight, preferably ≧90% by weight, of the saturation concentration of the metal halide is set in the phase (B) (described below). Here, the term “saturation concentration” is as defined in IUPAC: Compendium of Chemical Terminology, 2^{nd} edition (the “Gold Book”), compiled by A. D. McNaught and A. Wilkinson. Blackwell Scientific Publications, Oxford (1997).

In the case of repeated addition of the metal halide, the next addition in each case is carried out in such a way that a concentration of ≧70%, preferably ≧90%, of the saturation concentration of the metal halide is reestablished in the apparatus (V1), preferably in the phase (B). The next addition in each case of metal halide is thus carried out when the metal halide concentration has gone below the above limit values. In particular, the repeated addition of the metal halide is carried out in such a way that the abovementioned saturation-based concentrations of metal halide in the phase (B) are continually maintained. The next addition in each case of metal halide is thus carried out before the metal halide concentration has gone below the above limit values.

The continuous addition of the metal halide is preferably carried out in such a way that a concentration of ≧70%, preferably ≧90%, of the saturation concentration of the metal halide is continuously maintained in the apparatus (V1). In particular, this is maintained in the phase (B).

Furthermore, preference is given to the metal halide and the gaseous hydrogen halide (HX), preferably AlCl₃ and hydrogen chloride, being introduced simultaneously into the apparatus (V1).

In a further preferred embodiment of the present invention, the following phases are comprised in the apparatus (V1):

i) the phase (A) comprising the ionic liquid,
ii) the phase (B) comprising at least one hydrocarbon,
iii) optionally the phase (C) comprising solid metal halide, preferably solid AlX₃, and
iv) the phase (D) comprising gaseous HX.

The process of the invention, in particular the isomerization, is preferably carried out continuously. The compounds (products) formed in the chemical reaction, in particular in the isomerization, can be discharged from the apparatus (V1) by methods known to those skilled in the art.

For example, a stream comprising the phase (B) and the phase (A), with at least one hydrocarbon which was prepared in the chemical reaction being comprised in the phase (B), can be discharged from the apparatus (V1) in which the chemical reaction is carried out. This stream is in turn preferably introduced into a phase separation apparatus (phase separation unit). Phase separation apparatuses per se are known to those skilled in the art. This phase separation apparatus is preferably a phase separator.

The apparatus (V1) is preferably a reactor or a cascade of stirred vessels, and a phase separation apparatus, preferably a phase separator, is located downstream of the apparatus (V1). Furthermore, preference is given to the reactor or the cascade of stirred vessels and optionally the phase separation apparatus being coupled on the gas side.

Furthermore, the phase (A) comprising the ionic liquid is preferably separated off from the phase (B) comprising at least one hydrocarbon in the phase separation apparatus, with the phase (A) preferably being recirculated to the apparatus (V1), in particular to the reactor or to the starting point of the cascade of stirred vessels.

In the phase separation apparatus, preference is given to a first stream comprising at least 70% by weight, preferably at least 90% by weight, of the phase (A) and a second stream comprising at least 70%, preferably at least 90%, of the phase (B) being separated from one another. The above figures in % are based on the corresponding amounts comprised in the stream introduced into the phase separation apparatus.

Furthermore, preference is given, for the purposes of the present invention, to a contact apparatus (V2), which is preferably a moving bed, a fluidized bed or a stirred vessel, being installed upstream of the apparatus (V1), with the metal halide firstly being introduced into the contact apparatus (V2) and being conveyed from there into the apparatus (V1). The metal halide can be added in solid or liquid form, particularly preferably in solid form.

An apparatus (V3) for solid/liquid or liquid/liquid separation, which is preferably a phase separator, a gravity separator, a hydrocyclone, an apparatus having a dead-end filter or a cross-flow filter, can in turn be installed downstream of the contact apparatus (V2). The apparatus (V3) for solid/liquid or liquid/liquid separation is optionally integrated into the contact apparatus (V2), for example by (V2) being a stirred vessel which comprises a stirring zone and, arranged above this, a disengagement zone in which gravity-induced separation of solid and liquid takes place. A stream which is enriched in solid and has been separated off in the apparatus (V3) for solid/liquid or liquid/liquid separation is preferably recirculated to the contact apparatus (V2).

Regardless of the presence of a downstream apparatus (V3) for solid/liquid or liquid/liquid separation, preference is given in relation to the contact apparatus (V2) to the metal halide being added repeatedly or continuously to the contact apparatus (V2) by means of an apparatus for metering or conveying solid or liquid; in the case of solid, preferably by means of a star feeder or pneumatic transport; in the case of liquid, preferably by means of a pump.

Preference is likewise given to a liquid which comprises the materials to be reacted in the apparatus (V1) and/or which is fed into the apparatus (V1) flowing through the contact apparatus (V2).

In a preferred embodiment, the presence of a second, in particular solid, phase in the contact apparatus (V2) is continually monitored visually or by means of another suitable apparatus or method, preferably by means of a turbidity measurement, and when the second phase disappears, metal halide is introduced into the contact apparatus (V2) by means of an apparatus for metering or conveying solid.

In a preferred embodiment of the present invention, the recirculated phase (A) which originates from the above-described phase separation apparatus, in particular the phase separator, flows through the contact apparatus (V2) and (V2) is located between phase separation apparatus and apparatus (V1), with an apparatus (V3) for solid/liquid separation or liquid/liquid separation optionally being installed downstream of (V2).

For the purposes of the present invention, cyclohexane is preferably isolated from the output from the apparatus (V1), in particular from the hydrocarbon-comprising output from a phase separation unit, preferably a phase separator, installed downstream of the apparatus (V1). Methods and apparatuses for separating cyclohexane from such an output or stream, in particular when the output is a hydrocarbon mixture, are known to those skilled in the art. Further purification steps (for example a scrub using an aqueous and/or alkaline phase), which are likewise known to those skilled in the art, can optionally be carried out before the isolation of cyclohexane.

In FIG. 1, the process of the invention is again illustrated by a preferred embodiment which is preferably carried out as an isomerization. “MX” is metal halide, “f” means solid and “I” means liquid or dissolved. “IL” is ionic liquid. “AO” is a shut-off device which is connected to the reservoir (R). “PC” is a pressure measurement device which is connected to a control device in such a way that it acts on at least one pressure-influencing device (“actuator”). “A” denotes phase (A), with the respective main component of this phase being placed in parentheses (in the present case ionic liquid which has been initially placed in the apparatus (V1)). “B” denotes phase (B), while “KW1” denotes a first hydrocarbon mixture and “KW2” denotes a second hydrocarbon mixture which is formed in a chemical reaction, preferably an isomerization, from KW1 in the apparatus (V1).

The phase B (KW1) which is to be reacted and comprises at least one hydrocarbon is fed continuously into the apparatus (V1); a metal halide (MX) is optionally also introduced into (V1). Under operating conditions, (V1) comprises liquid reaction mixture and a gas phase which is in contact with this. (V1) is connected via the shut-off device (AO) to the reservoir (R) which comprises a gas which consists to an extent of more than 90 mol %, particularly preferably more than 98 mol %, of the hydrogen halide and has a pressure above the pressure of the gas phase over the reaction mixture. In (V1), a pressure measurement device which is connected to a regulating device is present in the gas phase. The totality of pressure measurement device and regulation device is in FIG. 1 denoted as (PC). (PC) controls the setting of the shut-off device (AO) in such a way that the shut-off device is opened when the pressure goes below a threshold value, while the shut-off device is closed when the pressure exceeds a threshold value. The detailed design of this regulating system can be effected in various ways known to those skilled in the art.

The present invention is illustrated below with the aid of the examples.

### General Experimental Conditions:

The experiments are carried out using the following substances and compositions:

Ionic liquid (A) having the composition (CH₃)₃NHAl₂Cl₇, a hydrocarbon mixture (B) having the components methylcyclopentane, cyclohexane, n-hexane and isohexane and additionally, for the example according to the invention, gaseous HCl. The ionic liquid will hereinafter also be referred to as “IL” and the hydrocarbon mixtures B and B1 as “organics” or “organic phase”.

The experimental arrangement is shown in FIG. 2.

The ionic liquid (CH₃)₃NHAl₂Cl₇ is placed in a 250 ml double-wall stirred reactor (V1) at 60° C. The hydrocarbon mixture (MCP, CH, n-hexane, isohexane) is metered with weighing control (30 g/h) and taken off again from a phase separator (PT) which is installed directly on the reactor. In the reactor (V1), the reaction of the hydrocarbon mixture, an isomerization of methylcyclopentane to cyclohexane, takes place. The isomerized hydrocarbon mixture is referred to as B1. The fill level of the reactor is regulated by adjustment of the variable overflow between V1 and PT. Here, a dispersion of B1 in A is fed to the phase separator in which the two phases are separated. The ionic liquid as heavier phase (A) is obtained as bottom phase and is conveyed by means of a pump back into the reactor (V1). The upper organic phase (B1) is taken off and its composition is determined by analysis by gas chromatography.

### Example 1

Composition of organic phase (B) at the beginning:

27% by weight of n-hexane
52% by weight of MCP
20% by weight of CH
1% by weight of i-hexanes
Reaction temperature: 60° C.
Fill quantity: 185 ml (257 g) of IL
Organics feed rate: 30 g/h
HCl feed rate: 20 standard ml/h (0-118 h)
40 standard ml/h (after 118 h)
Phase ratio of IL/organics ≈15 (V/V)
Stirrer: blade stirrer, speed of rotation=900 rpm

### Procedure

In this experiment, 20 standard ml/h of HCl gas are additionally metered by means of a gas burette into the gas space of the reactor. Based on the organics feed rate of 30 g/h, this corresponds to 0.1% by weight. After the experiment has been running for 118 h, the HCl addition rate is increased to 40 standard ml/h of HCl (corresponding to 0.2% by weight). Balance experiments have shown that the HCl gas which is metered into the gas space of the reactor dissolves very quickly in the organics. It can therefore be assumed that at least 95% of the HCl gas is dissolved into the liquid phase.

### Results

| Time for which experiment | |
|---|---|
| has been running [h] | Conversion of MCP [%] |
| 30 | 48 |
| 50 | 49 |
| 75 | 47 |
| 85 | 46 |
| 150 | 47 |
| 200 | 49 |
| 300 | 48 |

A constant MCP conversion can be achieved over a long period of time by contacting with gaseous HCl, and the rapid decrease in the activity as in the comparative example can thus be prevented.

### Comparative Example 2

Composition of organic phase (B) at the beginning:

27% by weight of n-hexane
52% by weight of MCP
20% by weight of CH
1% by weight of i-hexanes
Reaction temperature: 60° C.
Fill quantity: 185 ml (257 g) of IL
Organics feed rate: 30 g/h
Phase ratio of IL/organics ≈5 (V/V)
Stirrer: blade stirrer, rotational speed=900 rpm

### Results

| Time for which experiment | |
|---|---|
| has been running [h] | Conversion of MCP [%] |
| 10 | 50 |
| 50 | 47 |
| 75 | 45 |
| 115 | 42 |
| 135 | 40 |
| 160 | 32 |
| 180 | 28 |

After an initially high activity, the MCP conversion drops to below 30% within 180 hours.

1-17. (canceled)

18. A chemical reaction process of at least one hydrocarbon in an apparatus (V1) in the presence of an ionic liquid and a hydrogen halide (HX), wherein a liquid reaction mixture comprising at least one hydrocarbon, the hydrogen halide and the ionic liquid and a gas phase comprising the hydrogen halide are present in the apparatus (V1), with the liquid reaction mixture and the gas phase being in direct contact with one another and gaseous hydrogen halide being introduced into the apparatus (V1) in such a way that the hydrogen halide partial pressure in the gas phase is kept constant during the chemical reaction.

19. The process according to claim 18, wherein the hydrogen halide (HX) is hydrogen chloride.

20. The process according to claim 19, wherein the hydrogen chloride is dry hydrogen chloride.

21. The process according to claim 188, wherein the hydrogen halide partial pressure in the gas phase is in the range from 1.1 to 5 bara.

22. The process according to claim 188, wherein the ionic liquid comprises at least one metal component and at least one halogen component as anion or at least one metal halide is introduced into the apparatus (V1) during the chemical reaction.

23. The process according to claim 22, wherein the metal halide is introduced repeatedly or continuously into the apparatus (V1) or the halogen component and the metal component of the anion of the ionic liquid and the metal halide are the same.

24. The process according to claim 22, wherein
i) in the anion of the ionic liquid, the metal component is selected from among Al, B, Ga, In, Fe, Zn and Ti or the halogen component is selected from among F, Cl, Br and I, or
ii) the metal halide is selected from among AlX₃, BX₃, GaX₃, InX₃, FeX₃, ZnX₂ and TiX₄ where X=halogen.

25. The process according to claim 22, wherein the ionic liquid has a haloaluminate ion having the composition AlₙX₍₃ₙ₊₁₎ where 1<n<2.5 and X=halogen as anion and the ionic liquid has an ammonium ion as cation.

26. The process according to claim 25, wherein the ionic liquid has trialkylammonium as cation or a chloroaluminate ion of the composition AlₙCl₍₃ₙ₊₁₎ where 1<n<2.5 as anion.

27. The process according to claim 188, wherein the ionic liquid in the liquid reaction mixture in the apparatus (V1) comprises greater than 50% by weight of a phase (A) which has a higher viscosity than a phase (B) in which the at least one hydrocarbon is present to an extent of more than 50% by weight and the phases (A) and (B) are in direct contact with one another.

28. The process according to claim 18, wherein, in the apparatus (V1), the ionic liquid is used as catalyst and the hydrogen halide is used as cocatalyst in a chemical reaction.

29. The process according to claim 28, wherein the ionic liquid is used in an isomerization.

30. The process according to claim 188, wherein the apparatus (V1) is a reactor or a cascade of stirred vessels or a phase separation apparatus is located downstream of the apparatus (V1).

31. The process according to claim 30, wherein the phase (A) comprising the ionic liquid is separated off from the phase (B) comprising at least one hydrocarbon in the phase separation apparatus, with the phase (A) being recirculated to the apparatus (V1).

32. The process according to claim 31, wherein the phase (A) is recirculated to the reactor or to the starting point of the cascade of stirred vessels.

33. The process according to claim 30, wherein the reactor or the cascade of stirred vessels and optionally the phase separation apparatus are coupled on the gas side.

34. The process according to claim 22, wherein the metal halide and the gaseous hydrogen halide (HX) are introduced simultaneously into the apparatus (V1).

35. The process according to claim 34, wherein the metal halide is AlCl₃ and HX is hydrogen chloride.

36. The process according to claim 188, wherein the pressure in the apparatus (V1) is kept constant by using a two-point regulating system which acts on a shut-off device to a hydrogen halide reservoir.

37. The process according to claim 188, wherein the following phases are comprised in the apparatus (V1):
i) phase (A) comprising the ionic liquid,
ii) phase (B) comprising at least one hydrocarbon,
iii) optionally phase (C) comprising solid metal halide, and
iv) phase (D) comprising gaseous HX.

38. The process according to claim 188, wherein the hydrogen halide partial pressure in the gas phase is kept constant by regulating the pressure in the apparatus (V1) by repeated or continuous introduction of gaseous hydrogen halide into the apparatus (V1).

39. The process according to claim 188, wherein the gas phase in the apparatus (V1) is connected via a shut-off device to a reservoir, where the contents of the reservoir comprise at least 90 mol % of the hydrogen halide and the reservoir has a pressure which is greater than the hydrogen halide partial pressure of the gas phase in the apparatus (V 1).
